(19) United States
(12) Patent Application Publication
(10) Pub. No.: US 2013/0302395 A1
(43) Pub. Date: Nov. 14, 2013

(54) COATED ANTIMICROBIAL ARTICLES

(71) Applicant: 3M INNOVATIVE PROPERTIES COMPANY, St. Paul, MN (US)

(72) Inventors: Jeffrey F. Andrews, Stillwater, MN (US); Matthew T. Scholz, Woodbury, MN (US); Joseph A. Tucker, Shoreview, MN (US); Kaveh Pournoor, St. Paul, MN (US)

(21) Appl. No.: 13/931,460

(22) Filed: Jun. 28, 2013

Related U.S. Application Data
(63) Continuation of application No. 11/504,150, filed on Aug. 15, 2006, now abandoned.
Application No. 11/504,150 is a continuation of application No. 09/572,549, filed on May 17, 2000, now abandoned.
(60) Provisional application No. 60/135,271, filed on May 21, 1999.

(51) Int. Cl. A01N 25/08; A61L 31/16; A61L 26/00

(52) U.S. Cl. CPC A01N 25/08; A61L 26/0066; A61L 31/16
USPC 424/412; 424/411

(57) ABSTRACT

Antimicrobial articles that include a fatty acid monoester and an enhancer are described that are effective for killing at least 99.9% of microorganisms on the surface of the article.

### CROSS-REFERENCE TO RELATED APPLICATIONS

This application is a continuation of U.S. application Ser. No. 11/504,150, filed Aug. 15, 2006, now pending, which is a continuation of U.S. application Ser. No. 09/572,549, filed May 17, 2000, now abandoned, which claims priority to U.S. Provisional Application Ser. No. 60/135,271, filed May 21, 1999, the disclosure of which is incorporated by reference in their entirety herein.

### TECHNICAL FIELD

The invention relates to articles that include an antimicrobial composition. In particular, the invention relates to coated articles that include a fatty acid monoester and an enhancer.

### BACKGROUND

Antimicrobial nonwoven articles and other types of extruded materials have been prepared by incorporation of antimicrobial agents directly into a polymeric hot melt prior to extrusion. This method allows the antimicrobial agents to be directly incorporated into the nonwoven and migrate onto the surface. Durable antimicrobial articles are obtained from such processes due to the continuous migration over time of the antimicrobial to the surface of the article. Hot-melt processes, however, require very high temperatures, e.g., 300° C. or higher. At such temperatures, many antimicrobial agents, especially organic molecules, face problems with thermostability and volatility. Thus, alternative methods for incorporating antimicrobial agents into nonwoven materials are needed.

### SUMMARY

The invention is based on the discovery that synergistic combinations of fatty acid monoesters and enhancers have been found to have antimicrobial activity on dry or essentially dry articles. Articles that are coated with compositions containing such molecules and dried are self-disinfecting, i.e., microorganisms that come into contact with the surface of the article are killed. Fatty acid monoesters and enhancers useful in the invention generally are of low mammalian toxicity, allowing articles of the invention to be used in a wide variety of applications, including applications resulting in direct contact with food or direct contact with humans and animals.

In one aspect, the invention features an antimicrobial article including a coating. The coating includes a fatty acid monoester and an enhancer, wherein the fatty acid monoester and the enhancer are present in an amount effective to kill at least 99.9% of microorganisms, e.g., Gram positive or Gram negative bacteria, or viruses. In some embodiments, the fatty acid monoester and the enhancer are present in an amount effective to kill at least 99.99% of microorganisms. The fatty acid monoester can be glycerol monolaurate, glycerol monocaprylate, glycerol monocaprate, propylene glycol monolaurate, propylene glycol monocaprylate, or propylene glycol monocaprate. The enhancer can be a chelating agent, e.g., sodium acid pyrophosphate, acidic sodium hexametaphosphate, or ethylenediaminetetraacetic acid and salts thereof, or an organic acid, e.g., an organic acid selected from the group consisting of lactic acid, tartaric acid, adipic acid, succinic acid, citric acid, ascorbic acid, malic acid, mandelic acid, acetic acid, sorbic acid, benzoic acid, and salicylic acid. Lactic acid and mandelic acid are particularly useful enhancers. The coating further can include a surfactant.

The antimicrobial article can be a nonwoven material, a woven fabric or web material, a sponge material, a fibrous batt material, a foam material, a surgical drape or gown, a face mask, a wound dressing, a disposable diaper, filter media, a cast padding, a stockinette, a respirator, food packaging, dental floss, a sponge, a textile, or a wipe. The antimicrobial article can have increased hydrophilicity as compared with a corresponding article without the coating.

The invention also features a surgical drape or face mask that includes a coating, wherein the coating includes a fatty acid monoester and an enhancer, and wherein the fatty acid monoester and the enhancer are present in an amount effective to kill at least 99.9% of microorganisms. The coating can further include a surfactant.

In another aspect, the invention features a method of making an antimicrobial article. The method includes treating the article with an antimicrobial composition, wherein the composition includes 0.001 wt. % to 30 wt. % of a fatty acid monoester and 0.001 wt. % to 85 wt. % of an enhancer; and drying the treated article. The antimicrobial article is effective for killing at least 99.9% of microorganisms. The fatty acid monoester can preferably be present at 0.01 wt. % to 5.0 wt. % in the antimicrobial composition. The enhancer can preferably be present at 0.5 wt. % to 5.0 wt. % in the antimicrobial composition. The antimicrobial composition further can include 0.001 wt. % to 30 wt. % of a surfactant, e.g., 0.5 wt. % to 5.0 wt. % of the antimicrobial composition.

The invention also features an alternative method of making an antimicrobial article. The method includes treating the article with a first composition comprising 0.001 wt. % to 30 wt. % of a fatty acid monoester; treating the article with a second composition comprising 0.001 wt. % to 85 wt. % of an enhancer; and drying the treated article, wherein the antimicrobial article is effective for killing at least 99.9% of microorganisms. The fatty acid monoester can preferably be present at 0.01 wt. % to 5.0 wt. % in the first composition. The enhancer can preferably be present at 0.5 wt. % to 5.0 wt. % in the second composition. The first composition further can include 0.001 wt. % to 30 wt. % of a surfactant, e.g., 0.5 wt. % to 5.0 wt. % of the first composition.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### DETAILED DESCRIPTION

In the present invention, selected articles are coated with liquid compositions containing a fatty acid monoester and an enhancer. After drying, it has been discovered, surprisingly, that the articles possess synergistic antimicrobial activity. Dry or essentially dry articles of the invention reduce the bacterial load of a challenge by greater than 99.9%. In certain cases, the bacterial load of both Gram-positive and Gram-negative bacteria are reduced by 99.9%. In contrast, identical articles coated with liquid compositions containing only fatty acid monoester or an enhancer typically fail to reduce bacterial load by 99.9%, and in particular, fail to reduce Gram-positive and Gram-negative bacteria load by 99.9%.

Thus, antimicrobial articles of the invention include a coating having a fatty acid monoester and an enhancer present in an amount effective to kill at least 99.9% of microorganisms upon contact with the article. In some embodiments, at least 99.99% of microorganisms are killed. As used herein, “antimicrobial” or “antimicrobial activity” means that an article has antimicrobial activity as measured by the American Association of Textile and Color Chemists (AATCC) Test Method 100-1993 (AATCC Technical Manual, 1997, pp. 143 to 144). This test method involves exposing treated articles of the present invention at selected times and temperatures to known or readily available viable bacterial strains, such as Klebsiella pneumonia, Escherichia coli, Staphylococcus aureus, and Staphylococcus epidermidis in a culture medium. Typically, treated articles are exposed for about 24 hours at temperatures of about 22° C. to about 35° C. After a specific exposure time, treated and untreated control samples are neutralized and the number of bacteria are determined by standard microbiological techniques. By comparing the number of bacteria from the treated sample to the number of bacteria from the control (treated samples at time zero or an untreated sample), the percent reduction of bacteria attributable to the antibacterial treatment can be calculated.

As used herein “amount effective” means that the amounts of fatty acid monoester and enhancer, as a whole, provide a spectrum of antimicrobial activity sufficient to kill most microorganisms on the article. The antimicrobial activity preferably extends to viruses, fungi, and bacteria, including Gram-positive and Gram-negative bacteria, pathogenic or undesired bacteria such as bacteria known to cause or be associated with food poisoning in humans, and bacteria related to or associated with food spoilage. The concentrations or amounts of each of the components, when considered separately, do not kill as great a spectrum of pathogenic or undesired microorganisms or reduce the number of such microorganisms to an acceptable level. Thus, the components of the composition when used together provide a synergistic antimicrobial activity to the article when compared to the same components used alone and under the same conditions.

The capability to reduce a bacterial load preferably is maintained for a sustained period of exposure. For example, the antimicrobial articles of the present invention can have a shelf life of at least about one year, and, more preferably, of at least about two years. Without being bound by a particular mechanism, it is thought that the dry state of the article prevents reaction of the fatty acid monoester with other components. Liquid compositions of fatty acid monoesters and enhancers possess synergistic antimicrobial activity. Fatty acid monoesters, however, are inherently reactive, especially in the presence of enhancers such as organic acids or chelating agents. For example, the monoesters can hydrolyze in an aqueous medium to the corresponding fatty acid, transesterify with a hydroxy-containing enhancer (e.g., lactic acid), or transesterify with a hydroxy-containing solvent. As a result of these reactions, the antimicrobial activity of the liquid composition may be reduced and shelf life may be shortened to less than one year. As most articles of the invention are dry when packaged or stored, the articles are not in intimate contact with an aqueous liquid or other solvent vehicle. Consequently, the fatty acid monoester is much less subject to reactions such as hydrolysis or transesterification over time.

### Fatty Acid Monoesters and Enhancers

Fatty acid monoesters suitable for use in the invention are monoesters that generally are considered food grade, are generally recognized as safe (GRAS), and/or are FDA-cleared food additives. In particular, fatty acid monoesters derived from C₈ to C₁₂ fatty acids such as glycerol monoesters of lauric, caprylic, or capric acid and/or propylene glycol monoesters of lauric, caprylic, or capric acid are useful. Monoglycerides useful in the invention typically are available in the form of mixtures of unreacted glycerol, monoglycerides, diglycerides, and triglycerides. However, it is preferred to use materials that contain a high concentration (e.g., greater than about 85 wt. %, preferably greater than about 90 wt. %, and more preferably greater than about 92%) of monoglyceride. Examples of particularly useful commercially available materials include glycerol monolaurate (GML), available from Med-Chem Laboratories, East Lansing, Mich., under the trade name LAURICIDIN™, glycerol monocaprylate (GM-C8) and glycerol monocaprate (GM-C10) available from Riken Vitamin Ltd., Tokyo, Japan under the trade names POEM™ M-100 and POEM™ M-200, respectively, and those available from the Henkel Corp. of Germany under the trade name “MONOMULS™ 90 L-12”. Propylene glycol monocaprylate (PG-C8) and propylene glycol monocaprate (PG-C10) are available from Uniquema International, Chicago, Ill.

Suitable enhancers are organic acids and chelating agents. Preferably, the enhancers are food grade, GRAS approved, and/or FDA-cleared food additives. Organic acids can include, for example, lactic acid, tartaric acid, adipic acid, succinic acid, citric acid, ascorbic acid, malic acid, mandelic acid, acetic acid, sorbic acid, benzoic acid, and salicylic acid. Chelating agents can include, for example, sodium acid pyrophosphate, acidic sodium hexametaphosphate (such as SPORIX™ acidic sodium hexametaphosphate), and ethylenediaminetetraacetic acid (EDTA) and salts thereof. Lactic acid and mandelic acid are particularly useful.

### Preparing Articles of the Invention

Fatty acid monoester, enhancer, and other acceptable materials (e.g., emulsifiers) are dissolved, dispersed, and/or emulsified in a liquid vehicle such as water, alcohol, or propylene glycol to form a liquid composition. Non-limiting examples of emulsifiers include anionic and nonionic surfactants such as dioctyl sodium sulfosuccinate, sodium lauryl sulfate, acyl lactylates such as sodium lauroyl lactylate, sorbitan esters such as sorbitan monolaurate, dodecylbenzene sulfonate and its salts, polyglycerol esters such as decaglyceryl tetraoleate, and block copolymers of polyalkylene oxide, e.g., polyethylene oxide and polypropylene oxide, available as Pluronics™ and Tetronics™ from BASF. Dioctyl sodium sulfosuccinate is commercially available as GEMTEX™ SC40 surfactant (40% dioctyl sodium sulfosuccinate in isopropanol) from Finetex Inc., Spencer, N.C.

Typically, the fatty acid monoester is about 0.001 to 30 weight % and the enhancer is about 0.001 to 85 wt. % of the liquid composition used to prepare the article of the invention. For example, the fatty acid monoester can be 0.01 to 5.0 wt. % of the liquid composition and the enhancer can be about 0.5 to 5.0 wt. % of the liquid composition. Surfactants, when present, typically are 0.001 to 30 wt. % of the liquid composition and preferably, 0.5 wt. % to 5.0 wt. % of the liquid composition. The liquid composition may also include a liquid carrier or solvent, e.g., water or an alcohol. The liquid composition is coated on the article by applying the composition to a portion of or over the entire exterior surface of the article using conventional application techniques, such as dipping, spraying, printing, brushing, sponging, or padding at temperatures preferably ranging from about 10° C. to about 100° C. Alternatively, the article may be prepared by treating with a first liquid composition containing the fatty acid monoester and optionally a surfactant, and separately treated with a second liquid composition containing the enhancer. Preferably, the coated surface or surface portion of the article is fully wetted with the liquid composition. The liquid carrier or solvent, if present, then is removed from the article by, for example, drying in an oven or air drying, to provide an essentially dry coating of the enhancer material and monoester on the surface of the article. The percent dry solids on the article can range from about 4% to about 45%. As used herein, “dry coating,” “essentially dry coating,” “dry solids,” and the like, mean that the dried article contains a dry coating having at least about 50 wt. % solids, preferably at least about 75 wt. % solids, and more preferably at least about 95 wt. % solids. Antimicrobial articles prepared according to this method are effective to kill at least 99.9% of microorganisms on the surface of the article, and preferably are effective to kill at least 99.99% of microorganisms.

The fatty acid monoester and enhancer may be most conveniently applied as essentially solventless liquid or molten compositions with the optional addition of an acceptable carrier material. For skin contact articles, suitable carrier materials include emollients and humectants such as those described in U.S. Pat. No. 5,951,993. In addition, emollients such as oils (for example, hydrocarbons and alkyl esters) and skin acceptable alkyl alcohols and polyethoxylated alcohols, and combinations thereof, also may improve the skin feel of the coated articles. The molten compositions are generally applied to most articles at the lowest temperature required to keep the material suitably molten. This is typically about 50° C.-150° C. The compositions are applied to most non-woven, woven or knitted articles at a loading of 0.5-25 g/sq meter, more preferably 1-20 g/sq meter, and most preferably at about 2-10 g/sq meter.

Additionally, the fiber and yarn articles of this invention can be treated with the antimicrobial compositions in typical sizing-type operations. Oils and other lubricants may be added in order to ensure a good sizing.

### Characterization of Articles of the Invention

Articles of the present invention include synthetic and natural fibers or filaments. Suitable synthetic fibers, filaments, and yarns include, but are not limited to, polyolefins, polyesters, and polyamides such as nylons, polyurethanes, halogenated polyolefins, polyacrylates, polyureas, polyacrylonitriles, as well as copolymers and polymer blends. Suitable natural fibers include cotton, rayon, jute, hemp, and the like, which may be present as staple fibers or spun into yarns.

Articles of the invention also include various materials, including foam materials, nonwoven, woven, and knit fabrics or webs, fibrous batts, and sponges. The term “nonwoven web” or “nonwoven fabric” refers to a web or fabric having a structure of individual fibers that are interlaid in an irregular manner. In contrast, knit or woven fabrics have fibers that are interlaid in a regular manner. Preferable articles of the invention are made by spunbound and melt blown processes. “Spunbound” refers to small diameter fibers that are “spun” by extruding molten thermoplastic material in the form of filaments from a plurality of fine, usually circular, capillaries of a spinneret, and then rapidly reducing the diameter of the extruded filaments. See, for example, U.S. Pat. Nos. 4,340,563 and 3,692,618 for a description of spunbound methods. The filaments are bonded together by passage between the rolls of a heated calender. “Melt blown” refers to a process of extruding molten thermoplastic material through a plurality of fine, typically circular, die capillaries as molten threads or filaments into a high velocity, typically heated, gas stream (e.g., air), which reduces the diameter of the filaments and deposits the filaments on a collecting surface to form a web of randomly disbursed melt blown fibers.

Non-limiting examples of particular articles include single and multi-layer nonwoven constructions and wound dressings, medical drapes and gowns, disposable diapers, filter media, face masks (e.g., surgical masks), orthopedic cast padding/stockinettes, textiles, respirators, food packaging, dental floss, home and industrial wipes, and battery separators.

The invention may find particular utility as an antimicrobial face mask, e.g., a surgical mask, or as an antimicrobial medical drape or gown, e.g., a surgical drape. Face masks are used as barriers between the wearer and the environment, and are well described in the art, e.g., in U.S. Pat. No. Re. 28,102 (Mayhew). Through their filtration efficiency, face masks can remove particulates (organic, inorganic, or microbiological) from the incoming or outgoing breath. Face masks are generally not antimicrobially active even though they are commonly used in a health care setting as a method of minimizing pathogen transmission risk. The invention includes a face mask with antimicrobial activity, that is a mask capable of killing microorganisms that come into contact with it. This activity extends to antimicrobial kill of such common organisms like bacteria, fungi, and viruses, e.g., the influenza A virus and the rhinovirus, the cause of the common cold. Surgical drapes may be constructed from single layers of a fibrous web material or include multi-layered laminates that include one or more film layers, e.g., as described in U.S. Pat. No. 3,809,077 (Hansen) and U.S. Pat. No. 4,522,203 (Mays). Surgical drapes require sterilization prior to use and since the drapes generally do not have inherent antimicrobial activity, any microbial contamination can remain on the surface of these drapes. The invention includes surgical drapes that can be self-sterilizing through the application of an antimicrobial coating to the surface of the surgical drape. Active surfaces like the self-sterilizing surgical drapes of this invention can provide long term antimicrobial kill of microorganisms coming in contact with the drape surface. Antimicrobial articles of the invention may be more hydrophilic than corresponding articles not treated with a fatty acid monoester. Thus, preferred articles of the invention are capable of readily being wet with water and/or aqueous compositions. Often it is preferred that such hydrophilic articles are capable of absorbing at least 5 times their own weight with water. Absorbent, antimicrobial articles can advantageously be used as wound dressings because they are able to absorb large quantities of wound exudates and retard growth of bacteria in the absorbent layer, and, in some cases, in the wound. A further advantage of the articles is that the antimicrobial activity can reduce the sterilization load associated with the wound dressing when the device is sterilized (for example by exposure to ethylene oxide) prior to or after packaging. The invention will be further described in the following examples, which do not limit the scope of the invention described in the claims.

### EXAMPLES

### Glossary

### Monoesters

GML: glycerol monolaurate, available from Med-Chem Laboratories, East Lansing, Mich. under the trade name “LAURICIDIN™”.
GM-C8: glycerol monocaprylate, available as POEM™ M-100 from Riken Vitamin LTD, Tokyo, Japan.
GM-C10: glycerol monocaprate, available as POEM™ M-200 from Riken Vitamin LTD, Tokyo, Japan.
PG-C8: propylene glycol monocaprylate, available from Uniquema International, Chicago, Ill.
PG-C10: propylene glycol monocaprate, available from Uniquema International, Chicago, Ill.

### Surfactant

SC40: GEMTEX™ SC40 surfactant (40% dioctyl sodium sulfosuccinate in isopropanol) commercially available from Finetex Inc., Spencer, N.C.

### Enhancer Materials

LA: Lactic acid, USP, commercially available from J.T. Baker, Phillipsburg, N.J.
MA: Mandelic acid, USP, commercially available from Avocado Research Chemicals, Ltd., Heysham, UK.
SA: Salicylic acid, USP, commercially available from J.T. Baker.

### Substrates

PAD-1: absorbent pads commonly used in the bottom of meat and poultry packages as absorbent pads; obtained from Cub food store, St. Paul, Minn.
PAD-2: commercially available paper (cellulose-based, available from Great Lakes Tissue Co., Cheboygan, Mich.) as used in multiple layers to make absorbent pads in the packaging of meat and poultry.
PAD-3: 100% cotton cloth.
PAD4: toilet paper (commercially available as 2-Ply White Tissue #0780500 Scott Surpass JRT Junior Jumbo Roll Bath Tissue from Kimberly Clark Corporation).
MASK: surgical masks made of nonwoven polypropylene BMF (commercially available from 3M Company, St. Paul, Minn., Product No. 1818).
DRAPE-1: Surgical drapes made of thermally laminated polyethylene film and nonwoven polypropylene BMF commercially available from 3M Company, St. Paul, Minn. as BIOCADE™ brand surgical drapes.
DRAPE-2: Polypropylene BMF surgical drape commercially available from 3M Company, St. Paul, Minn. as PREVENTION™ brand surgical drape.

### Test Methods

Melt-Blown Extrusion Procedure: A process similar to that described in Wente, Superfine Thermoplastic Fibers, 48 INDUS. ENG G CHEM. 1342 (1956), or in Wente et al., MANUFACTURE OF SUPERFINE ORGANIC FIBERS (Naval Research Laboratories Report No. 4364, 1954), was used for the preparation of nonwoven webs of this invention. Unless otherwise stated, the basis weight of the resulting webs was 60±5 g/m² (GSM), and the desired diameter of the microfibers was 7 to 12 micrometers. The width of the web was about 12 inches (30.5 cm). Unless otherwise stated, the extrusion temperature was 255° C., the primary air temperature was 258° C., the pressure was 124 kPa (18 psi), with a 0.076 cm air gap width, and the polymer throughput rate was about 180 g/hr/cm.

Antimicrobial Test: The materials of this invention were cut into 3.8 cm×3.8 cm square samples (unsterilized) and evaluated for antimicrobial activity according to the American Association of Textile and Color Chemists (AATCC) Test Method 100-1993, as published in the AATCC Technical Manual, 1997, Pages 143-144. Modifications to the test method included the use of Tryptic Soy Broth as the nutrient broth and for all dilutions, and Tryptic Soy Agar as the nutrient agar. Letheen Broth (VWR Scientific Products, Batavia, Ill.) was used as the neutralizing solution.

Water Absorbency: Evaluation of the water absorbency of certain nonwoven polypropylene articles of this invention was measured using the following test procedure. A 3.8-cm×3.8-cm sample was weighed, dipped into 22° C. deionized water for ten seconds, and then removed from the water holding a corner of the pad with the smallest possible area. The excess water was allowed to drip off from one corner until no additional water was freely dripping. The sample was then weighed again. The sample percent absorbency was then calculated using the formula: Absorbency (%)=(Wet Sample Weight−Dry Sample Weight)×100±Dry Sample Weight. Results reported are the average of ten replications.

### Examples 1 to 3 and Comparative Examples C1 to C3

Multiple samples of a nonwoven polypropylene BMF web (basis weight 63 g/m²) prepared using the Melt Blown Extrusion Procedure with Exxon 3866 polypropylene (Exxon Chemical Co., Baytown, Tex.) were dipped in an isopropanol solution of 1.0% GML and 1.0% of various enhancer materials (i.e., lactic acid (LA), salicylic acid (SA), and mandelic acid (MA)).

The samples were submerged in the isopropanol solution for approximately 10 seconds, removed, air dried, and evaluated for antimicrobial efficacy against Staphylococcus aureus (a Gram-positive bacterium) and against Klebsiella pneumoniae (a Gram-negative bacterium) using the Antimicrobial Test. The results (each data point representing a single run) are shown in Table 1. Example 3 was also evaluated for antimicrobial efficacy against Staphylococcus epidermidis (a Gram-positive bacterium). In each evaluation of an Example, a coated web was compared to a control of an uncoated web. Negative numbers indicate an increase from the baseline bacterial count.

TABLE 1
% Reduction of Bacteria Colony Forming Units (CFU)

| | Sample | | 1-Hour Exposure | | | 24-Hour Exposure | | | |
|---|---|---|---|---|---|---|---|---|---|
| Ex. | (wt. %) | Bacteria | 10° C. | 25° C. | 35° C. | 10° C. | 25° C. | | 35° C. |
| 1 | GML (1.0%), LA (1.0%) | Staph. aureus | 88.75 | — | 99.90 | 100.00 | — | | — |
| | (Control-Uncoated Pad) | | | | (14.29) | (4.17) | | | |
| 2 | GML (1.0%), SA (0.5%) | | — | 99.52 | — | — | 100.00 | (−5323) | — |
| | (Control-Uncoated Pad) | | | (20.34) | | | | | |
| 3 | GML (1.0%), MA (1.0%) | | — | 99.99 | — | — | 99.99 | (−163.2) | — |
| | (Control-Uncoated Pad) | | | (−5.26) | | | | | |
| C1 | GML (1.0%) | | — | −32.27 | — | — | 99.99 | (−387.5) | — |
| | (Control-Uncoated Pad) | | | (−15.9) | | | | | |
| C2 | LA (1.0%) | | — | −4.05 | — | — | −156.76 | (−656.8) | — |
| | (Control-Uncoated Pad) | | | (−35.1) | | | | | |
| C3 | MA (1.0%) | | — | 32.43 | — | — | 99.87 | (−656.8) | — |
| | (Control-Uncoated Pad) | | | (−35.1) | | | | | |
| 1 | GML (1.0%), LA (1.0%) | Kleb. pneum. | 90.04 | — | 94.98 | 100.00 | — | | — |
| | (Control-Uncoated Pad) | | (−1.22) | | (78.45) | (10.98) | | | |
| 2 | GML (1.0%), SA (0.5%) | | — | 99.91 | — | — | 98.82 | (−2264) | — |
| | (Control-Uncoated Pad) | | | (−10.0) | | | | | |
| 3 | GML (1.0%), MA (1.0%) | | — | 80.71 | — | — | 100.00 | (99.82) | — |
| | (Control-Uncoated Pad) | | | (−94.1) | | | | | |
| C1 | GML (1.0%) | | — | 40.81 | — | — | −10981 | (−12062) | — |
| | (Control-Uncoated Pad) | | | (8.11) | | | | | |
| C2 | Lactic Acid (1.0%) | | — | 67.96 | — | — | −3686.4 | (−2716) | — |
| | (Control-Uncoated Pad) | | | (64.08) | | | | | |
| C3 | MA (1.0%) | | — | 90.59 | — | — | 63.05 | (−2757) | — |
| | (Control-Uncoated Pad) | | | (63.55) | | | | | |
| 3 | GML (1.0%), MA (1.0%) | Staph. epider. | — | 81.41 | — | — | 99.99 | (−263.2) | — |
| | (Control-Uncoated Pad) | | | (7.35) | | | | | |

The data show that only the combination of an effective amount of GML and an enhancer material provided high antimicrobial activity to both Gram-negative and Gram-positive bacteria.

### Examples 4 to 11

In these Examples, the antimicrobial efficacy of various fibrous substrates coated with GML and various enhancer materials (and optionally surfactant SC40) were evaluated. Multiple 3.8 cm×3.8 cm samples of the coated test materials were dipped for about 10 seconds in either ethanol solutions (Examples 4 to 5), isopropanol solutions (Examples 6 to 8), or aqueous dispersions containing the SC40 surfactant (Examples 9 to 11). The samples were air dried and evaluated for antimicrobial activity according to the Antimicrobial Test using Staphylococcus aureus and Klebsiella pneumoniae. The sample materials, compositions of the solutions and aqueous dispersions, and results of the Antimicrobial Test (each data point representing a single run) are summarized in Table 2. In each instance, the treated samples were compared with an untreated control. In Examples involving the surgical mask, the entire mask was dipped into the isopropanol solution or aqueous dispersion and, after drying, 3.8 cm×3.8 cm samples were cut for use in the Antimicrobial Test. Negative numbers indicate an increase in the bacterial count from the baseline.

The data show that all examples provided at least 99.9% control of both Gram-negative and Gram-positive bacteria when tested at 25° C. to 35° C. with a 24-hour exposure time. All but one sample provided at least 99.99% control of both types of bacteria at these conditions.

TABLE 2
% Reduction of Bacteria CFU

| | | | 1-Hour Exposure | | | 24-Hour Exposure | | | |
|---|---|---|---|---|---|---|---|---|---|
| Ex. | Sample | Bacteria | 10° C. | 25° C. | 35° C. | 10° C. | 25° C. | 35° C. | |
| 4 | PAD-1 | Staph. aureus | 84.32 | | 99.92 | 100.00 | | 100.00 | (−5080) |
| | GML (2.0%), LA (4.0%) | | (−5.41) | | (15.38) | (−18.92) | | | |
| | (Control-Uncoated) | Kleb. pneum. | 93.55 | | 41.33 | 99.89 | | 100.00 | (−3900) |
| | | | (1.32) | | (21.33) | (10.53) | | | |
| 5 | PAD-2 | Staph aureus | 69.74 | | 99.41 | 99.99 | | 100.00 | (−996.1) |
| | GML (2.0%), LA (4.0%) | | (91.03) | | (92.45) | (11.79) | | | |
| | (Control-Uncoated) | Kleb. pneum. | 97.38 | | 100.00 | 100.00 | | 100.0 | (−4819) |
| | | | (4.26) | | (−170.16) | (26.74) | | | |
| 6 | PAD-3 | Staph aureus | | 56.64 | | | 99.92 | | |
| | GML (2.0%), LA (4.0%) | | | (23.01) | | | (−422.1) | | |
| | (Control-Uncoated) | Kleb. pneum. | | 60.71 | | | 100.00 | | |
| | | | | (−14.29) | | | (−569.64) | | |
| 7 | PAD-4 | Staph aureus | | | 100.00 | | | 100.00 | (−10481) |
| | GML (1.0%), LA (4.0%) | | | | (−11.63) | | | | |
| | (Control-Uncoated) | Kleb. pneum. | | | 100.00 | | | 100.00 | (−962.3) |
| | | | | | (−233.33) | | | | |
| 8 | MASK | Staph. aureus | | 81.16 | | | 100.00 | | |
| | GML (1.0%), MA (1.0%) | | | (−10.96) | | | (−253.42) | | |
| | (Control-Uncoated) | Kleb. pneum. | | 99.13 | | | 99.99 | | |
| | | | | (56.23) | | | (−4050.94) | | |
| 9 | MASK | Staph. aureus | | 100.00 | | | 100.00 | | |
| | GML (1.0%), MA (1.0%) | | | (−39.47) | | | (−226.32) | | |
| | SC40 (5.0%) | Kleb. pneum. | | 99.96 | | | 100.00 | | |
| | (Control-Uncoated) | | | (69.64) | | | (−2971.43) | | |
| 10 | DRAPE-1 | Staph aureus | | 100.00 | | | 100.00 | | |
| | GML (1.0%), LA (1.5%) | | | (−12.24) | | | (24.49) | | |
| | SC40 (5.0%) | Kleb. pneum. | | 93.52 | | | 100.00 | | |
| | (Control-Uncoated) | | | (33.33) | | | (−3603.70) | | |
| 11 | DRAPE-2 | Staph aureus | | 100.00 | | | 100.00 | | |
| | GML (1.0%), LA (1.5%) | | | (89.33) | | | (−23.33) | | |
| | SC40 (5.0%) | Kleb. pneum. | | 99.52 | | | 100.00 | | |
| | (Control-Uncoated) | | | (36.90) | | | (−2995.24) | | |

### Examples 12 to 13

A 3.8-cm×3.8-cm×0.7-cm sample of a polyurethane open-cell foam material (POLYCRIL™ 400 with carrier and backing films removed by hand, Fulflex, Middleton, R.I.) was placed in a glass jar containing an isopropanol solution of 1.0% GML and 1.6% LA. The jar was capped and shaken for about 2 minutes, and the sample was removed and dried for 72 hours in a ventilation hood. The sample was weighed before and after contact with the test solution, and % Dry Solids was calculated as follows: Dry Weight (after coating)−Dry Weight (before coating)×100±Dry Weight (after coating). The results (three replications) are reported in Table 3 as Example 12. A similarly coated sample of the foam material was evaluated for antimicrobial efficacy against Staphylococcus aureus and Staphylococcus epidermidis (both Gram-positive bacterium), and against Pseudomonas aeruginosa (a Gram-negative bacterium) using the Antimicrobial Test (test exposure times of 1 hour and 24 hours, both at 34-35° C.). The results (each data point representing a single run) are also shown in Table 3. In each antimicrobial evaluation, a coated foam sample was compared to a control of an uncoated foam sample. Negative numbers indicate an increase from the baseline bacterial count.

Example 12 was repeated except that the coating composition (in place of the isopropanol solution) was an aqueous composition of GML (1.5%), LA (1.5%), SC40 surfactant (5.0%), and deionized water. The results of % Dry Solids and antimicrobial testing are provided in Table 3 as Example 13.

TABLE 3
% Dry Solids Coating Weight and % Reduction of Bacteria CFU

| | Nonwoven Sample | % Dry | S. aureus | | S. epidermis | | P. aeruginosa | |
|---|---|---|---|---|---|---|---|---|
| Ex. | (Coating Comp.) | Solids | 1-Hr | 24-Hr | 1-Hr | 24-Hr | 1-Hr | 24-Hr |

| | | | | | | | | |
|---|---|---|---|---|---|---|---|---|
| 12 | GML (1.0%), LA | 13.6 | 14.02 | 98.98 | 71.56 | 100 | 44.18 | 100 |
| | (1.6%) | | (−33.6) | (−21.7) | (24.8) | (−206) | (70.6) | (−5221) |
| | (Control-Uncoated) | | | | | | | |
| 13 | GML (1.5%), LA | 42.6 | 100 | 100 | 9 9.18 | 99.99 | 99.24 | 99.46 |
| | (1.5%) SC40 (5.0%) | | (−33.6) | (−21.7) | (24.8) | (−206) | (70.6) | (−5221) |
| | (Control-Uncoated) | | | | | | | |

The data from Table 3 show that both coated foam samples (Examples 12 and 13) provided high antimicrobial activity (generally at least about 99% bacteria reduction, and often at least about 99.99% bacteria reduction, after 24 hours exposure at 34-35° C.) to all three bacteria species.

### Examples 14 to 25

Samples (3.8-cm×3.8-cm) of a nonwoven polypropylene BMF web (basis weight 63 g/m²) prepared using the Melt Blown Extrusion Procedure with Exxon 3866 polypropylene (Exxon Chemical co., Baytown, Tex.) were dipped in an isopropanol solution containing a fatty acid monoester (GML or PG-C8) and various enhancer materials (LA or MA). The samples were submerged in the isopropanol solution for approximately 10 seconds, removed, and air-dried. The samples were weighed before and after contact with the test solution, and % Dry Solids was calculated as described above. The results (ten replications) are reported in Table 4 as Examples 14-18. The samples were also evaluated for antimicrobial efficacy against Staphylococcus aureus (a Gram-positive bacterium) and against Escherichia coli (a Gram-negative bacterium) using the Antimicrobial Test (test exposure times of 1 hour and 24 hours, both at 22-23° C.). The results (each data point representing a single run) are also reported in Table 4. In each antimicrobial evaluation, a coated nonwoven sample was compared to a control of an uncoated nonwoven sample. Negative numbers indicate an increase from the baseline bacterial count.

Additional experiments were conducted in the same manner as Examples 14-18, except that the coating composition (in place of the isopropanol solution) was an aqueous composition of fatty acid monoester, enhancer, SC40 surfactant, and deionized water. The results of % Dry Solids and antimicrobial testing are provided in Table 4 as Examples 19-25.

TABLE 4
% Dry Solids Coating Weight and % Reduction of Bacteria CFU

| | Nonwoven Sample | % Dry | S. aureus | | E. coli | |
|---|---|---|---|---|---|---|
| Ex. | (Coating Comp.) | Solids | 1-Hr | 24-Hr | 1-Hr | 24-Hr |
| 14 | GML (0.1%), LA (1.5%) | 4.9 | 85.92 | 100 | 68.56 | 64.55 |
| | (Control-Uncoated) | | (−1.97) | (−8518) | (18.73) | (−3645) |
| 15 | GML (0.1%), MA (1.5%) | 9.5 | 75.68 | 100 | 93.01 | −267.2 |
| | (Control-Uncoated) | | (3.28) | (−249.7) | (25.78) | (−4587) |
| 16 | GML (1.0%), LA (1.5%) | 13.5 | 95.44 | 100 | 70.13 | −2140 |
| | (Control-Uncoated) | | (25.73) | (−742.1) | (37.34) | (−3764) |
| 17 | GML (1.0%), MA (1.5%) | 17.8 | 99.14 | 100 | 99.99 | 100 |
| | (Control-Uncoated) | | (78.61) | (−229.5) | (50.93) | (−3596) |
| 18 | PG-C8 (1.0%), LA (1.5%) | 10.9 | 95.32 | 99.88 | 99.87 | 18.99 |
| | (Control-Uncoated) | | (25.73) | (−742.1) | (37.34) | (−3764) |
| 19 | GML (0.1), LA (1.5%) | 20.9 | 99.93 | 100 | 54.85 | −2275 |
| | SC40 (5.0%) | | (−1.97) | (−8518) | (18.73) | (−3645) |
| | (Control-Uncoated) | | | | | |
| 20 | GML (0.1%), MA (1.5%) | 29.9 | 100 | 100 | 99.91 | 100 |
| | SC40 (5.0%) | | (3.28) | (−249.7) | (25.78) | (−4587) |
| | (Control-Uncoated) | | | | | |
| 21 | GML (0.1%), LA (3.0%) | 27.7 | 100 | 100 | 72.08 | 100 |
| | SC40 (5.0%) | | (25.73) | (−742.1) | (37.34) | (−3764) |
| | (Control-Uncoated) | | | | | |
| 22 | GML (1.0%), LA (1.5%) | 29.0 | 98.42 | 100 | 8.59 | −3611 |
| | SC40 (5.0%) | | (3.28) | (−249.7) | (25.78) | (−4587) |
| | (Control-Uncoated) | | | | | |
| 23 | GML (1.0%), LA (3.0%) | 28.6 | 99.99 | 100 | 78.26 | −1794 |
| | SC40 (5.0%) | | (78.61) | (−229.5) | (50.93) | (−3596) |
| | (Control-Uncoated) | | | | | |
| 24 | GML (1.0%), MA (1.5%) | 34.3 | 100 | 100 | 99.97 | 100 |
| | SC40 (5.0%) | | (78.61) | (−229.5) | (50.93) | (−3596) |
| | (Control-Uncoated) | | | | | |
| 25 | PG-C8 (1.0%), LA (1.5%) | 25.8 | 99.98 | 100 | 77.92 | −1247 |
| | SC40 (5.0%) | | (25.73) | (−742.1) | (37.34) | (−3764) |
| | (Control-Uncoated) | | | | | |

The data from Table 4 show that all of the coated nonwoven samples (Examples 14-25) provided high antimicrobial activity (generally at least about 99.9% bacteria reduction, after 24 hours exposure at 22-23° C.) to at least one of the two bacteria species. Several samples (e.g., Examples 17, 20, 21, and 24) provided 100% reduction of both S. aureus and E. coli bacteria.

### Examples 26 to 40

Samples (3.8-cm×3.8-cm) of a nonwoven polypropylene BMF web (basis weight 63 g/m²) prepared using the Melt Blown Extrusion Procedure with Exxon 3866 polypropylene were coated with a fatty acid monoester and an enhancer material as described in Examples 14-25. Certain samples (Examples 26-31) were dipped in isopropanol solutions and other samples (Examples 32-40) were dipped in an aqueous composition that included the SC40 surfactant. All samples were evaluated for antimicrobial efficacy against Staphylococcus aureus and against Escherichia coli using the Antimicrobial Test, and % Dry Solids were calculated. The results (each data point representing a single run) are provided in Table 5. In each antimicrobial evaluation, a coated nonwoven sample was compared to a control of an uncoated nonwoven sample. Negative numbers indicate an increase from the baseline bacterial count.

TABLE 5
% Dry Solids Coating Weight and % Reduction of Bacteria Colony Forming Units
(CFU)

| | Foam Sample | % | S. aureus | | E. coli | |
|---|---|---|---|---|---|---|
| Ex. | (Coating Comp.) | Dry Solids | 1-Hr | 24-Hr | 1-Hr | 24-Hr |
| 26 | GML (0.1%), LA | 9.4 | 92.18 | 100 | −202.6 | −1374 |
| | (3.0%) (Control- | | (21.33) | (−833.3) | (18.95) | (44.74) |
| | Uncoated) | | | | | |
| 27 | GML (1.0%), MA | 25.8 | 99.10 | 100 | 99.67 | 100 |
| | (3.0%) | | (21.33) | (−833.3) | (18.95) | (44.74) |
| | (Control-Uncoated) | | | | | |
| 28 | GML (0.1%), MA | 21.7 | 100 | 100 | 100 | 100 |
| | (3.0%) | | (17.27) | (−1394) | (41.69) | (−6239) |
| | (Control-Uncoated) | | | | | |
| 29 | PG-C8 (1.0%), MA | 23.8 | 100 | 100 | 100 | 100 |
| | (3.0%) | | (17.27) | (−1394) | (41.69) | (−6239) |
| | (Control-Uncoated) | | | | | |
| 30 | PG-C8 (1.0%), MA | 17.3 | 100 | 100 | 100 | 98.90 |
| | (1.5%) | | (17.27) | (−1394) | (41.69) | (−6239) |
| | (Control-Uncoated) | | | | | |
| 31 | PG-C8 (1.0%), LA | 19.6 | 99.99 | 100 | 100 | 100 |
| | (3.0%) | | (9.27) | (−818.9) | (−9.90) | (−4431) |
| | (Control-Uncoated) | | | | | |
| 32 | GML (1.0%), MA | 38.1 | 100 | 100 | 97.00 | 100 |
| | (3.0%) | | (21.33) | (−833.3) | (18.95) | (44.74) |
| | SC40 (5.0%) | | | | | |
| | (Control-Uncoated) | | | | | |
| 33 | GML (0.1%), MA | 34.3 | 100 | 100 | 100 | 100 |
| | (3.0%) | | (21.33) | (−833.3) | (18.95) | (44.74) |
| | SC40 (5.0%) | | | | | |
| | (Control-Uncoated) | | | | | |
| 34 | GML (0.1%), MA | 19.5 | 99.64 | 100 | 52.20 | −1815 |
| | (0.25%) | | (17.27) | (−1394) | (41.69) | (−6239) |
| | SC40 (5.0%) | | | | | |
| | (Control-Uncoated) | | | | | |
| 35 | PG-C8 (1.0%), MA | 31.5 | 100 | 100 | 100 | 100 |
| | (1.5%) | | (17.27) | (−1394) | (41.69) | (−6239) |
| | SC40 (5.0%) | | | | | |
| | (Control-Uncoated) | | | | | |
| 36 | PG-C8 (1.0%), MA | 38.6 | 100 | 100 | 100 | 100 |
| | (3.0%) | | (9.27) | (−818.9) | (−9.90) | (−4431) |
| | SC40 (5.0%) | | | | | |
| | (Control-Uncoated) | | | | | |
| 37 | PG-C8 (1.0%), LA | 27.2 | 100 | 100 | 90.49 | 100 |
| | (3.0%) | | (9.27) | (−818.9) | (9.90) | (−4431) |
| | SC40 (5.0%) | | | | | |
| | (Control-Uncoated) | | | | | |
| 38 | GM-C8 (1.0%, LA | 33.1 | 99.95 | 100 | 96.74 | 100 |
| | (1.5%) | | (9.27) | (−818.9) | (−9.90) | (−4431) |
| | SC40 (5.0%) | | | | | |
| | (Control-Uncoated) | | | | | |
| 39 | PG-C10 (1.0%), LA | 24.9 | 97.97 | 99.98 | 52.08 | −2582 |
| | (1.5%) | | (9.27) | (−818.9) | (−9.90) | (−4431) |
| | SC40 (5.0%) | | | | | |
| | (Control-Uncoated) | | | | | |
| 40 | GM-C10 (1.0%), LA | 28.1 | 99.95 | 100 | 94.90 | −1202 |
| | (1.5%) | | (9.27) | (−818.9) | (−9.90) | (−4431) |
| | SC40 (5.0%) | | | | | |
| | (Control-Uncoated) | | | | | |

The data from Table 5 show that all of the coated nonwoven samples (Examples 26-40) provided high antimicrobial activity (generally at least about 99.9% bacteria reduction, after 24 hours exposure at 22-23° C.) to at least one of the two bacteria species. All but one sample (Example 39) provided 100% reduction of Staphylococcus aureus bacteria and all but five samples (Examples 26, 30, 34, 39, and 40) provided 100% reduction of Escherichia coli bacteria at these conditions.

### Example 41

This Example illustrates the degree of water absorbency of the treated nonwoven web constructions of this invention as compared to an untreated control web.

A sample (3.8-cm×3.8-cm) of a nonwoven polypropylene BMF web (basis weight 63 g/m²) prepared using the Melt Blown Extrusion Procedure with Exxon 3866 polypropylene was treated with an aqueous composition of GML (1.0%), LA (1.5%), SC40 surfactant (5.0%), and deionized water as described in Examples 19-25.

The amount of water absorbed and the percent water absorbency of the sample (Example 41) was measured according to the Water Absorbency Method described above. Results are provided in Table 6 and are compared to the results of testing an identical nonwoven sample that had not been treated. These data show that the treated sample was highly water absorbent, absorbed over eight times its own weight with water, and absorbed over ten times as much water as the untreated sample.

TABLE 6

| | Components | Dry | Water | |
|---|---|---|---|---|
| | of Treatment | Sample | Absorbed | Water Absorbency |
| Example | Composition | Weight (g) | (g) | (%) |
| 41 | GML (1.0%), | 0.150 | 1.22 | 813 |
| | LA (1.5%), | | | |
| | SC-40 (5.0%) | | | |
| Control | None | 0.105 | 0.114 | 108 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-27. (canceled)

28. An antimicrobial article comprising a substrate and a coating, said coating comprising a fatty acid monoester and an enhancer, wherein said fatty acid monoester and said enhancer are present in an amount effective to kill at least 99.9% of microorganisms contacted with the article, wherein the substrate is selected from the group consisting of a wipe, a face mask, a respirator, a surgical drape, a surgical gown, a wound dressing, and a food packaging.

29. The antimicrobial article of claim 28, said coating further comprising a surfactant.

30. The antimicrobial article of claim 28, wherein the substrate comprises synthetic fibers or filaments, natural fibers or filaments, yarns, foam materials, nonwovens, wovens, knit fabrics, or webs.

31. The antimicrobial article of claim 28, wherein the coating comprises 0.001 wt. % to 30 wt. % of a fatty acid monoester and 0.001 wt. % to 85 wt. % of an enhancer.

32. The antimicrobial article of claim 28, wherein the substrate is a wipe.

33. The antimicrobial article of claim 28, wherein the substrate is a face mask or a respirator.

34. The antimicrobial article of claim 28, wherein the substrate is a surgical drape or a surgical gown.

35. The antimicrobial article of claim 28, wherein the substrate is a wound dressing.

36. The antimicrobial article of claim 28, wherein the coating comprises 0.01 wt. % to 5.0 wt. % of a C8 to C12 fatty acid monoester of glycerol and/or a propylene glycol, wherein said fatty acid monoester comprises greater than about 85 wt. % monoglyceride; and 0.5 wt. % to 5.0 wt. % of an enhancer selected from the group consisting of a chelating agent and an organic acid, wherein said organic acid is selected from the group consisting of lactic acid, tartaric acid, adipic acid, succinic acid, citric acid, ascorbic acid, malic acid, mandelic acid, acetic acid, sorbic acid, benzoic acid, salicylic acid, and combinations thereof.

37. The antimicrobial article of claim 28, wherein the article is self-disinfecting.

38. A method of making an antimicrobial article according to claim 28 comprising:
treating a substrate with a fatty acid monoester and an enhancer to create a treated substrate, wherein said fatty acid monoester and said enhancer are present in an amount effective to kill at least 99.9% of microorganisms contacted with the article, and wherein the substrate is selected from the group consisting of a wipe, a face mask, a respirator, a surgical drape, a surgical gown, a wound dressing, and a food packaging; and
drying said treated substrate until the treated substrate is dry or essentially dry to create the antimicrobial article.

39. The method of claim 38, wherein treating the substrate comprises treating the substrate with a single composition comprising 0.001 wt. % to 30 wt. % of a fatty acid monoester and 0.001 wt. % to 85 wt. % of an enhancer.

40. The method of claim 38, wherein treating the substrate comprises a first treating step of treating the substrate with a first composition comprising 0.001 wt. % to 30 wt. % of a fatty acid monoester; and a second treating step of treating the substrate with a second composition comprising 0.001 wt. % to 85 wt. % of an enhancer.

41. The method of claim 38, wherein treating the substrate comprises treating the substrate with an antimicrobial composition, wherein said composition comprises: 0.01 wt. % to 5.0 wt. % of a C8 to C12 fatty acid monoester of glycerol and/or a propylene glycol, wherein said fatty acid monoester comprises greater than about 85 wt. % monoglyceride; and 0.5 wt. % to 5.0 wt. % of an enhancer selected from the group consisting of a chelating agent and an organic acid, wherein said organic acid is selected from the group consisting of lactic acid, tartaric acid, adipic acid, succinic acid, citric acid, ascorbic acid, malic acid, mandelic acid, acetic acid, sorbic acid, benzoic acid, salicylic acid, and combinations thereof; and
drying said treated article comprises drying the treated article to form a dry or essentially dry coating comprising at least 50 wt. % solids based on the total weight of the dry coating.

42. A method of killing microorganisms that come into contact with an article selected from a wipe, a face mask, a respirator, a surgical drape, a surgical gown, or a wound dressing comprising contacting the microorganisms with the article, wherein the article comprises a substrate and a coating, said coating comprising a fatty acid monoester and an enhancer, wherein at least 99.9% of microorganisms are killed when contacted with the article.
